(19) United States
(12) Patent Application Publication
(10) Pub. No.: US 2007/0060838 A1
(43) Pub. Date: Mar. 15, 2007

(54) Biopsi and injection catheters

(76) Inventor: Nabil Dib, Phoenix, AZ (US)

Correspondence Address: SQUIRE SANDERS & DEMPSEY LLP, TWO RENAISSANCE SQUARE, 40 NORTH CENTRAL AVENUE, SUITE 2700, PHOENIX, AZ, 85004-4498, US

(21) Appl. No.: 11/258,644

(22) Filed: Oct. 24, 2005

Related U.S. Application Data
(63) Continuation-in-part of application No. 10/820,183, filed on Apr. 6, 2004.

(51) Int. Cl. A61B 10/00

(52) U.S. Cl. 600/565; 600/562

(57) ABSTRACT

A medical device includes a needle and optionally a catheter and a sheath. The device takes a biopsy from a body tissue and/or injects a substance into a body tissue, such as heart tissue. One or more ports are located on the needle to either take a biopsy and/or inject a substance. If a catheter is included as part of the device, a relative motion restrictor may be included to restrict relative movement during needle deployment between the catheter and the sheath. Additionally, a driver may be employed to deploy the needle with one or more of a predefined force, acceleration and velocity to assist in needle penetration.

### FIELD OF THE INVENTION

The present invention relates to medical devices, and, more particularly, to a medical device for taking a biopsy from a patient and/or injecting a fluid into a patient.

### BACKGROUND OF THE INVENTION

Catheters are widely used today in variety of medical procedures, such as for medical treatment of the heart. U.S. Pat. No. 6,689,103, which is incorporated herein by reference, discloses an exemplary catheter.

FIG. 1 represents a well-known technique to access the heart 16 of a patient 12 with a catheter 10. Specifically, catheter 10 is typically inserted through an incision into a patient's artery 14 and fed into heart 16 for treatment, which may include, among other things, taking a biopsy of heart tissue for analysis and/or injecting fluid into heart tissue.

FIGS. 2A-2C illustrate a problem commonly encountered with such treatments. FIG. 2A shows a working end of catheter 10 in contact with heart tissue of heart 16. FIGS. 2B and 2C show a needle 18 being deployed from catheter 10 for insertion into the heart tissue. Oftentimes, and undesirably, the resistance of the heart tissue forces catheter 10 away from the heart tissue as needle 18 is deployed, as represented by the arrows in FIGS. 2B and 2C, and prevents insertion of needle 18 into the heart tissue.

Potentially exacerbating this problem is the relative ease by which catheter 10 may slide along needle 18, the flexibility of catheter 10 and the roughened

Potentially exacerbating this problem is the relative ease by which catheter 10 may slide along needle 18, the flexibility of catheter 10 and the roughened texture and increased density of damaged heart tissue, as may result from fibrosis, including the formation of scar tissue following a heart attack. The structure of catheters for obtaining heart-tissue biopsies also contributes to the problem. For capturing a biopsy sample, such catheters 10 typically have a dull needle tip and/or some other structure that is inherently resistive, such as an abrasive surface, a pincher or the like, however, by having such structures located on the tip of needle 18, resistance often is elevated to the point of inhibiting smooth needle entry into the heart tissue. Also contributing to the problem is the current manner in which needle 18 is deployed, typically by a mechanism, such as a plunger external to patient 12, which may be moved by manual force, a solenoid, hydraulic pressure or pneumatic pressure. However deployed, needle 18 is generally moved too slowly and with insufficient force for easy insertion into the heart tissue. Additionally, while some devices either collect a biopsy of body tissue or inject a substance into body tissue, it would be advantageous to have a single device that could both collect a biopsy and inject a substance into a body tissue.

### SUMMARY OF THE INVENTION

A medical device for taking a biopsy of body tissue and/or injecting a substance into body tissue is disclosed. As used herein, “body tissue” refers to any tissue of the body and a medical device according to the invention could be used for any suitable body tissue of any type of animal. The medical device includes a needle having one or more ports configured to collect a biopsy and one or more ports configured to inject a substance into the body tissue. One or more of the ports may be configured to both collect a biopsy and inject a substance into the body tissue. In one preferred embodiment the needle has a single port formed in the needle's side wall wherein the port is configured to collect a biopsy and inject a substance into the body tissue. As used herein, substance refers to any material, and is preferably a fluid, that can be injected into a body tissue. In a single procedure a device according to the invention could be used to either collect a biopsy, inject a substance or both.

The device may simply comprise a needle, which can take a biopsy of body tissue and/or, inject a substance into body tissue, and the needle is any structure suitable for this purpose. The device may also include a catheter in which case the needle is preferably attached to the distal end of the catheter. Most preferably, the needle is fully retained in a first position in the catheter lumen until being injected into the body tissue, at which point the needle moves into its second position wherein it is at least partially outside of the catheter lumen. The needle includes one or more ports for obtaining a biopsy and/or injecting a substance. The one or more ports may be positioned on the side wall of the needle and/or in the tip of the needle.

Both the foregoing general description and the following detailed description are exemplary and explanatory only and are not restrictive of the invention as claimed.

### BRIEF DESCRIPTION OF THE DRAWINGS

FIG. 1 illustrates a well-known technique to access the heart of a patient with a catheter.

FIGS. 2A-2C illustrate a problem encountered when employing a catheter to take a biopsy of tissue and/or to inject fluid into tissue wherein a needle has difficulty penetrating dense tissue such as heart muscle.

FIG. 3 is a simplified block diagram of a system including a medical device for taking a biopsy of tissue and/or injecting fluid into tissue, in accordance with the present invention.

FIG. 4 is a simplified flowchart illustrating a method of employing a medical device for taking a biopsy of tissue and/or injecting fluid into tissue, in accordance with the present invention.

FIGS. 5A-5E show cross-sectional views of a working (or distal) end of a medical device for taking a biopsy of tissue and/or injecting fluid into tissue, in accordance with the present invention.

FIG. 6 is a cross-sectional view of the distal end of the medical device of FIGS. 5A-5E.

FIG. 7 shows an alternate embodiment of a needle that can be used with the invention.

FIG. 8 shows an alternate embodiment of a needle that can be used with the invention.

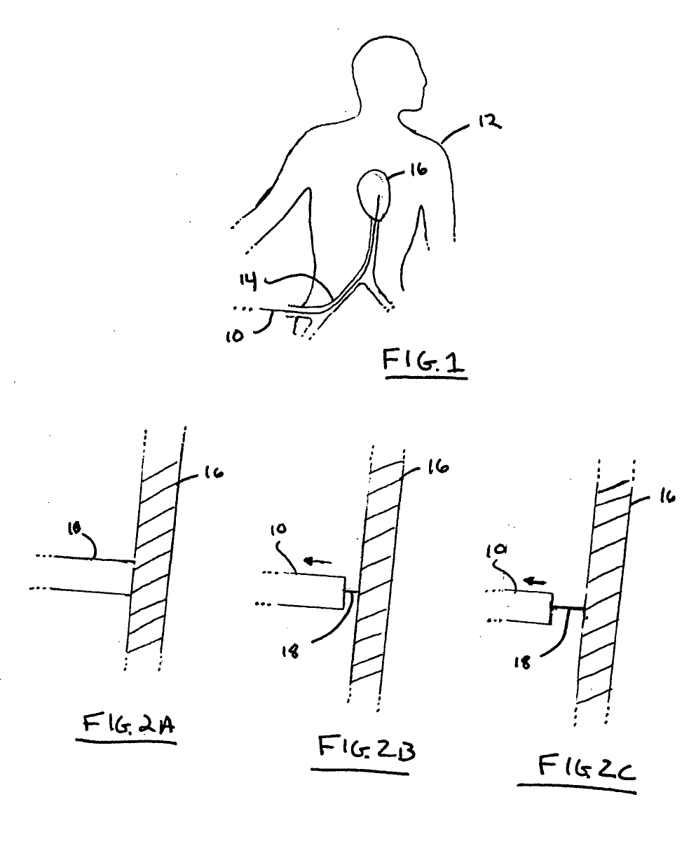

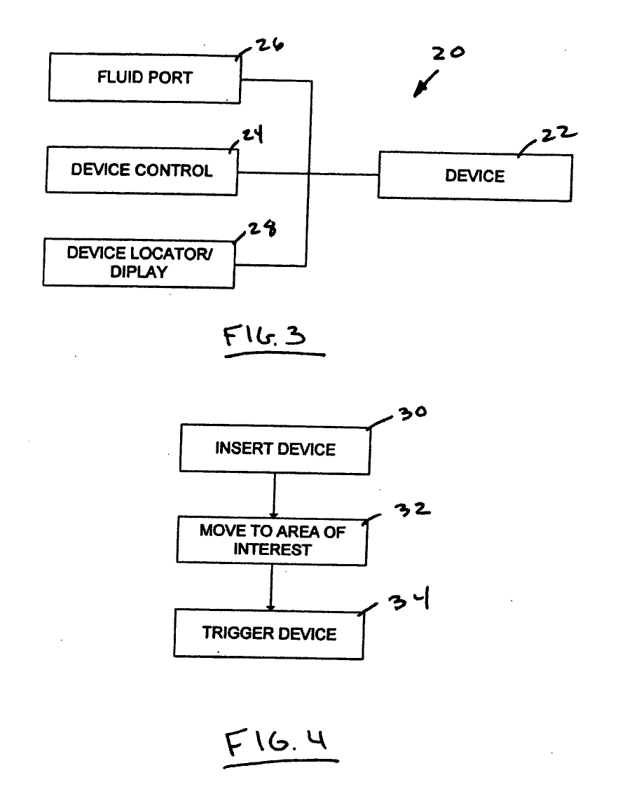

### DESCRIPTION OF THE EMBODIMENTS

Reference will now be made in detail to the present exemplary embodiments of the invention, examples of which are illustrated in the accompanying drawings.

Referring to FIG. 3, a medical system 20 may be utilized for performing any desired medical procedure. In an exemplary embodiment, medical system 20 may be employed to take a biopsy of and/or inject a fluid into a tissue, such as heart tissue. Medical system 20 may include one or more medical devices 22 (hereafter “medical device 22”), one or more medical device control systems 24 (hereafter “control system 24”), one or more fluid ports 26 (hereafter “port 26”) and/or one or more medical device locator and display systems 28 (hereafter “display system 28”).

Medical device 22 is any structure suitable for taking a biopsy and/or injecting a substance (particularly a fluid) into a body tissue. An injected substance may include any material for the desired procedure, for example, cells or genes.

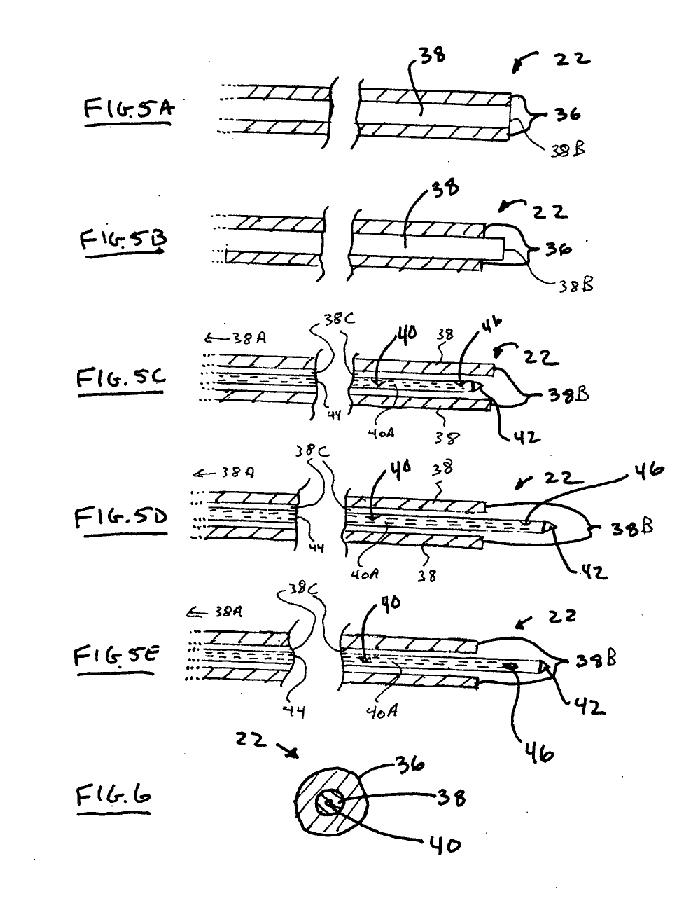

In one embodiment, medical device 22 comprises a catheter 38 and a needle 40, as shown in FIGS. 5A-5E (needle 40 is not shown in FIGS. 5A and 5B and sheath 36 is not shown in FIGS. 5C-5E for drawing simplification). Catheter 38 is any catheter of material, size and structure suitable for purposes of either taking a biopsy and/or injecting a substance into a body tissue utilizing a needle according to the invention. Catheter 38 typically has a working end (or distal end) 38A (not shown in the drawings although its relative position is indicated), and a proximal end 38B which is usually juxtaposed an operator (typically a medical professional) performing the desired medical procedure. Catheter 38 further comprises a catheter lumen 38C. In the embodiment shown, needle 40 is positioned at the distal end 38B of catheter 38. Needle 40 can be attached to catheter 38 in any suitable manner and as used in this context, “attached to” means that needle 40 also could be formed as part of catheter 38.

Needle 40 is any structure of suitable material, size and configuration for the purpose of collecting a biopsy of body tissue and/or injecting a substance. Needle 40 comprises a pointed tip 42, a side wall 40A, a needle lumen 44, and one or more ports 46. As shown, needle 40 is a cylindrical, tubular structure but it could have any cross-sectional shape suitable for taking a biopsy and/or injecting a substance. In one embodiment, needle 40 is a 26 or 27 gauge needle. Needle 40 could run the entire length of catheter 38 (if used with catheter 38) or needle 40 could not run the entire length of catheter 38 and instead be moved by a device in lumen 38C in a manner known by persons skilled in the relevant art.

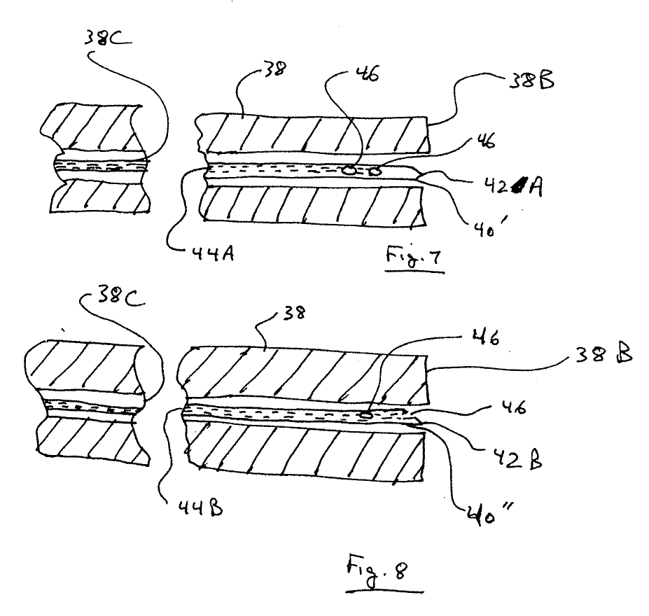

Pointed tip 42 is tapered and could be pointed in any manner sufficient to penetrate the body tissue for which needle 40 is being used. Port 46 is shown positioned in side wall 40A and can be used to collect (or receive) a biopsy of body tissue and/or to inject a substance into body tissue. Additionally, needle 40 may include more than one port. If needle 40 has more than one port, as shown in FIGS. 7-8, at least one port would be configured to receive a biopsy of body tissue and at least one port would be configured to inject a substance. Further, one or all of the ports could be configured to both receive a biopsy and inject a substance.

If needle 40 is used with catheter 38, needle 40 preferably has a first position (shown in FIG. 5C) wherein needle 40 is at least partially retained in, and preferably fully retained in, catheter 38. Needle 40 also has a second position, shown in FIGS. 5D and 5E, wherein tip 42 and port 46 extend outside of distal end 38B of catheter 38. In this case, needle 40 extends 2 mm to 10 mm beyond the distal end 38B of catheter 38 when moved to its second position. One way in which needle 40 is used is for it to be retained in its first position while catheter 38 is moved through the body of patient 12 and to be moved to its second position for use in a desired medical procedure, wherein it is injected into body tissue in order to collect a biopsy and/or inject a substance. Needle 40 or any needle used with a catheter according to the invention is moved either directly or indirectly by the operator.

Alternate needle embodiments are shown in FIGS. 7 and 8. In FIG. 7 a needle 40′ has a pointed tip 42A, a needle lumen 44A, and two ports 46A. At least one of the ports 46A can collect a biopsy and at least one can inject a substance. Alternatively, one or both of ports 46A can both collect a biopsy and inject a substance. Using such a structure the operator could potentially select which port(s) from which to collect a biopsy and/or inject a substance and the operator may select multiple ports or select a port that is, for example, positioned further into the body tissue or that is closer to the surface of the body tissue.

In FIG. 8 a needle 40″ has a pointed tip 42B, a needle lumen 44B and two ports 46B. In this embodiment, one of the ports is formed at tip 42B. At least one of the ports 46B can collect a biopsy and at least one can inject a substance. Alternatively, one or both of ports 46B can both collect a biopsy and inject a substance. Using such a structure the operator could select which port(s) from which to collect a biopsy and/or inject a substance and the operator may select a port that is positioned further into the body tissue or that is closer to the surface of the body tissue.

Needles 40′ and 40″ could each have a first position and a second position as previously described for needle 40.

A needle guide (not shown), such as a guide wire, is typically connected to needle 40 opposite pointed end 42 and extends through catheter lumen 38C to an operator performing the desired medical procedure. The needle guide is any device or structure that allows the operator to operate the needle for the desired medical procedure.

Referring to needle 40, the needle collects a biopsy due to tissue pressing into one or more ports 46 when needle tip 42 and port 46 are injected into the body tissue. The tissue presses into the port due to the natural tendency of the body tissue to press in against the needle after the needle is inserted into the body tissue. Each of the one or more ports that collects a biopsy preferably may have a sharp outer edge and as the needle retracts from the body tissue it severs a small biopsy. Alternatively, a small sliding window (or cutter), which is not shown, may be positioned on or over the outside surface of the needle or (most preferably) in the needle lumen and an operator can retract the cutter to expose the port and allow body tissue to enter the port and then extend the window to its closed position wherein it closes the port and in doing so severs the body tissue leaving a biopsy in the port. The cutter is preferably manually retracted and extended by the operator by pulling or pushing a device (such as a wire) that moves the cutter, and with which the cutter may be integrally formed. If a cuter is used the outer edge of a port associated with the cutter may or may not have a sharp, outer edge.

A substance is injected into the body tissue through one or ports 46 in needle 40. If a port 46 through which a substance is to be injected has a cutter sheath as described above, cutter is moved to its second, open position before the substance is injected. If a port is configured solely to inject a substance it need not have a cutter and/or a sharp outer edge since its function would not be to collect a biopsy.

Each of the needles 40, 40′ or 40″ described herein and any needle according to the invention could function in the manner described above, or in any suitable manner, to collect a biopsy and/or inject a substance.

In another embodiment, medical device 22 comprises catheter 38, needle 40 and a sheath 36, as best seen in FIGS. 5A-5E, (needle 40 is not shown in FIGS. 5A and 5B and sheath 36 is not shown in FIGS. 5C-5E for purposes of drawing simplification). Sheath 36 may comprise any sheath of material and size suitable for purposes of the desired medical procedure.

To permit loading a substance into device 22 for injection into patient 12, needle 40 has one or more ports 26. Port 26 is typically located outside of patient 12 during a medical procedure, where it is accessible to the operator. Port 26 is in fluid communication with one or more ports 46 in needle 40, preferably through the needle lumen. As previously described, port or ports 46 may be located anywhere on needle 40, including, as shown in FIGS. 5C-5E, one or more delivery ports 46. Thus, a substance loaded into port 26 may be injected into body tissue at port 46. Similarly, device 22 may be employed to take a biopsy of body tissue, such as body tissue in patient 12, at port 46, and the biopsy may be transported (preferably by vacuum) through the needle lumen for removal through port 26.

Any control system 24 may be employed that is suitable for controlling medical device 22, port 26 and/or display system 28, as desired for the selected medical procedure. Control system 24 may employ manual control and/or automatic control. Manual control typically employs a user to control, for example, move, an object, either directly, (wherein the operator touches the object to control it), or indirectly, (wherein the operator touches an intermediary structure to control the desired object). Automatic control typically employs one or more programs which, when executed, perform programmed operations to control, e.g., move, the desired object. Whether manual control and/or automatic control is employed to control medical device 22, port 26 and/or display system 28 for the selected medical procedure, any power source, such as manual, electric, electromechanical, hydraulic, pneumatic and the like, may be utilized to supply force to control, e.g., move the selected object as desired.

To control sheath 36, any sheath controller and/or sheath-control technique may be used to insert, move and/or in any way employ sheath 36 to perform the desired medical procedure. To control catheter 38, any catheter controller and/or catheter-control technique may be used to insert, move and/or in any way employ catheter 38 to perform the desired medical procedure. FIGS. 5A and 5B together illustrate an exemplary controlled movement of sheath 36 and/or catheter 38. More specifically, FIG. 5A shows coplanar alignment of the working ends of sheath 36 and catheter 38. FIG. 5B shows a changed alignment, which may result from movement of sheath 36 and/or catheter 38. For example, a medical professional rendering the desired medical procedure may, by moving sheath 36 and/or catheter 38, vary the “unsheathed” length, if any, of catheter 38 that may extend outside of sheath 36. Reducing the “unsheathed” length of catheter 38, i.e., covering more of catheter 38 with sheath 36, may tend to constrain or limit any potential bowing of catheter 38 that, in the absence of sheath 36, may otherwise occur during needle deployment toward an intended target of tissue. Reducing catheter bowing may tend to improve the ease with which needle 40 may enter body tissue.

Control system 24 may also selectively restrict relative movement between sheath 36 and catheter 38, which may include the sliding of catheter 38 within the lumen of sheath 36. Any structure that is suitable for this purpose may be employed. For example, a mechanical stop or an electromechanical stop (hereafter inclusively “stop”) may be used to selectively restrict relative movement between sheath 36 and catheter 38, e.g., restricting any movement, such as the sliding of catheter 38 within the lumen of sheath 36.

A stop may comprise a mechanical structure for, at a selected time, restricting the relative movement between sheath 36 and catheter 38, e.g., a clamp that may be selectively engaged to hold together a portion of sheath 36 and a portion of catheter 38 with some predefined force. A stop may also comprise an electromechanical structure for, at a selected time, restricting the relative movement between sheath 36 and catheter 38, e.g., one or more selectively-expandable regions on sheath 36 and/or catheter 38 that may be expanded to restrict relative motion between sheath 36 and catheter 38 during, for example, needle deployment toward an intended target of tissue. Control of a stop, whatever its structure, may be manual and/or automatic. Considering automatic control of a stop, for example, at a predetermined time, e.g., before or during needle deployment toward an intended target of tissue, the stop may automatically activate to restrict the relative movement between sheath 36 and catheter 38 for a predetermined period of time during needle deployment and then release the applied restriction against relative movement between sheath 36 and catheter 38 at a predetermined time, e.g., after needle 40 returns to within catheter 38.

Increased resistance to relative movement between sheath 36 and catheter 38, e.g., the sliding of catheter 38 within the lumen of sheath 36, may tend to prevent undesirable movement of catheter 38 away from target tissue that, in the absence of such resistance, may otherwise occur during needle deployment toward an intended target of tissue. Limiting movement of catheter 38 away from target tissue during needle deployment may tend to improve the ease with which needle 40 may enter tissue.

Needle 40 may also be controlled manually and/or automatically. Regardless of the manner of control, needle 40 may be driven to provide one or more different movements to perform the desired medical procedure. Such movements may include: 1) movement of needle 40 within the lumen of catheter 38, without extending needle 40 outside of the working end of catheter 38; 2) deploying needle 40, i.e., moving a portion of needle 40 to extend outside of the working end of catheter 38; 3) returning needle 40 within the lumen of catheter 38; 4) rotating needle 40 along its axis; 5) opening port 46; 6) closing port 46; and 7) any other desired movement.

In the case of manual needle control, for example, an operator rendering the desired medical procedure may depress a needle actuator, which may initiate and apply a manual force or other type of force to produce a desired needle movement, e.g., deploying needle 40. An opposing force may be provided by, for example, an opposing spring-loaded mechanism in the needle actuator, which may return needle 40 back within catheter 38 when the needle actuator is released. In the case of automatic needle control, for example, an operator rendering the desired medical procedure may position the medical device 22 in a position that automatically triggers a predefined operation of needle 40. Alternatively, once the operator confirms that medical device 22 is in the desired position, using, for example, display system 28, the operator may depress a needle actuator, causing needle 40 to perform an operation.

By way of example, a needle operation may include: 1) deploying needle 40 to enter tissue; 2) opening port 46, either before or after needle 40 enters the tissue to, for example, permit injecting a fluid or taking a biopsy; 3) rotating needle 40 along its axis, either before or after needle 40 enters the tissue to, for example, facilitate taking a biopsy; 4) returning needle 40 within the lumen of catheter 38; and 5) closing port 46 while needle 40 is within tissue to, for example, facilitate taking a biopsy, or after needle 40 is removed from the tissue. When deploying needle 40, a driver may be employed to selectively move needle 40 with a predefined force, a predefined acceleration, and/or a predefined velocity, to improve the ability of needle 40 to easily penetrate the tissue.

Display system 28 locates and displays medical device 22 within patient 12. If automatic triggering is used based on the location of medical device 22, display system 28 may provide such location information. Display system 28 employs any structure and/or method suitable for such purposes, and may be a system of the type described in either U.S. Pat. No. 6,902,528 entitled “Method and Apparatus for Magnetically Controlling Endoscopes in Body Lumens and Cavities,” U.S. Pat. No. 6,834,201 entitled “Catheter Navigation within a MR Imaging Device,” U.S. Pat. No. 6,817,364 entitled “Magnetically Navigated Pacing Leads, and Methods for Delivering Medical Devices,” or disclosed in U.S. Pat. Nos. 6,786,219; 6,755,816 or 6,702,804. The respective disclosures of these patents that are not inconsistent with the text of this application are hereby incorporated by reference.

A sensor 42 may be located on medical device 22, for example, on needle 40, and in communication with display system 28 to provide location information for medical device 22 back to display system 28. Sensor 42 may comprise any sensor that is suitable for this purpose and may be compatible with electrical and/or magnetic fields.

FIG. 4 illustrates a method of employing medical device 22 for taking a biopsy of tissue and/or injecting fluid into tissue. At step 30, an operator inserts medical device 22 into patient 14 using any technique. At step 32, the operator moves medical device 22 to an area of interest using any technique and may use display system 28 to monitor this process. At step 34, medical device 22 may be triggered to perform the desired medical operation, such as injecting a fluid into or taking a biopsy from tissue. Triggering may be automatic or manually selected.

FIGS. 5A-5B together illustrate an exemplary controlled movement of sheath 36 and/or catheter 38. More specifically, FIG. 5A shows coplanar alignment of the working ends of sheath 36 and catheter 38. FIG. 5B shows a changed alignment, which may result from movement of sheath 36 and/or catheter 38. Needle 40 is not shown in FIGS. 5A and 5B for purposes of drawing simplification.

Referring to FIGS. 5C-5E, sheath 36 is not shown for purposes of drawing simplification. FIG. 5C shows needle 40 within a lumen of catheter 38. FIG. 5D shows needle 40 being deployed from catheter 38, as represented by the arrow pointing away from the proximal end of catheter 38. As shown in FIG. 5D, port 46 may be closed during needle deployment, however, port 46 may be opened during needle deployment, after needle deployment, after the needle is injected into body tissue or at any time suitable for the selected medical procedure. FIG. 5E shows needle 40 with port 46 in an open position to facilitate, for example, the taking of a biopsy. At a suitable time, port 46 may be closed and needle 40 may be returned within the lumen of catheter 38, as represented by FIG. 5C.

FIG. 6 is a planar view from the front of the working end of medical device 22. As shown, sheath 36, catheter 38 and needle are coaxial, however, they need not be aligned.

Other embodiments of the invention will be apparent to those skilled in the art from consideration of the specification or practice of the invention disclosed herein. The specification and examples are thus exemplary only, with the true scope and spirit of the invention being set forth in the following claims and legal equivalents thereof.

What is claimed is:

1. A medical device comprising:
a catheter having a proximal end, a distal end and a catheter lumen; and
a needle attached to the distal end of the catheter, the needle having a pointed tip for injection into body tissue and one or more ports for collecting a biopsy of a body tissue and one or more ports are injecting a substance into the body tissue.

2. The device of claim 1 wherein the needle has one port configured to both collect a biopsy of body tissue and inject a substance into body tissue.

3. The device of claim 1 wherein the needle has a plurality of ports and at least one of the ports is configured to both collect a biopsy of body tissue and inject a substance into the body tissue.

4. The device of claim 1 wherein the needle has at least two ports and at least one port is configured to collect a biopsy of body tissue and at least one port is configured to inject a substance into the body tissue.

5. The device of claim 1 wherein the catheter has a catheter lumen and the needle is retained at least partially in the catheter lumen when it is not injected into the body tissue.

6. The device of claim 1 wherein the needle is comprised of nitrile.

7. The device of claim 1 wherein the needle is comprised of stainless steel.

8. The device of claim 1 wherein the needle comprises a needle lumen in communication with the port and in communication with the catheter lumen.

9. The device of claim 1 that further includes a vacuum source applied to the port.

10. The device of claim 1 wherein the needle is 2″ or less in length.

11. The device of claim 1 wherein the needle is either 26 gauge or 27 gauge.

12. The device of claim 1 wherein the needle has a first position wherein it is inside the catheter lumen and a second position wherein the pointed tip and port are outside of the catheter lumen, the needle moving from the first position to the second position in order to penetrate the body tissue.

13. The device of claim 1 wherein the catheter includes a structure that shears the biopsy material as the port retracts into the catheter lumen.

14. The device of claim 1 wherein the port has an outer opening defined by a sharp outer edge.

15. The device of claim 1 wherein the needle further includes an outer side wall and each of the one or more ports is positioned in the outer side wall.

16. The device of claim 1 wherein the port is in the pointed tip of the needle.

17. The device of claim 1 wherein the needle further includes an outer side wall and there is at least one port in the tip of the needle and at least one port in the outer side wall.

18. The device of claim 16 wherein at least one of the ports is configured to inject a substance into a body tissue.

19. The device of claim 16 wherein at least one of the ports is configured to collect a biopsy of body tissue.

20. The medical device of claim 1 further including a sheath having a sheath lumen through which the catheter is selectively moved.

21. The medical device of claim 19 further including means for selectively restricting relative movement between the sheath and the catheter.

22. The medical device of claim 1 further including a driver for selectively moving the needle with one or more of a predefined force, a predefined acceleration and a predefined velocity to penetrate the material.

23. The medical device of claim 21 wherein the driver selectively rotates the needle.

24. The medical device of claim 1 further including a sensor on the device for providing signals for determining position of the device.

25. The medical device of claim 1 wherein the sensor is detachable in an electrical or magnetic field.

26. The medical device of claim 23 wherein the sensor is on the needle.

27. The medical device of claim 1 further including a second port in fluid communication with the first port to retrieve the biopsy material from the first port.

28. The medical device of claim 1 wherein the needle includes a door that has a first position wherein it at least one covers at least one of the one or more ports and a second position wherein it does not cover the at least one of the one or more ports, the door being moved to the second position to allow a biopsy of body tissue to enter the port or to inject fluid into the body tissue.

29. A medical device comprising a needle having a pointed tip for injection into body tissue and one or more ports for collecting a biopsy of a body tissue and one or more ports for injecting a substance into the body tissue.

30. The device of claim 29 wherein the needle has one port configured to both collect a biopsy of body tissue and inject a substance into body tissue.

31. The device of claim 29 wherein the needle has a plurality of ports and at least one of the ports is configured to both collect a biopsy of body tissue and inject a substance into the body tissue.

32. The device of claim 29 wherein the needle has at least two ports and at least one port is configured to collect a biopsy of body tissue and at least one port is configured to inject a substance into the body tissue.

33. The device of claim 29 wherein the catheter has a catheter lumen and the needle is retained at least partially in the catheter lumen when it is not injected into the body tissue.

34. The device of claim 29 wherein the needle is comprised of nitrile.

35. The device of claim 29 wherein the needle is comprised of stainless steel.

36. The device of claim 29 wherein the needle comprises a needle lumen in communication with the port and in communication with the catheter lumen.

37. The device of claim 29 that further includes a vacuum source applied to the port.

38. The medical device of claim 30 wherein the needle includes a door that has a first position. Wherein it at least one covers at least one of the one or more ports and a second position wherein it does not cover the at least one of the one or more ports, the door being moved to the second position to allow a biopsy of body tissue to enter the port or to inject fluid into the body tissue.

39. The medical device of claim 28 wherein the needle is either 26 gauge or 27 gauge.
